(19) United States
(12) Patent Application Publication
(10) Pub. No.: US 2025/0134705 A1
(43) Pub. Date: May 1, 2025

(54) Breathable Thermal Contact Pad

(71) Applicant: C. R. Bard, Inc., Franklin Lakes, NJ (US)

(72) Inventors: Madeline Stich, Thornton, CO (US); Michael R. Hoglund, Windsor, CO (US); Gabriel A. Johnston, Broomfield, CO (US); Sean E. Walker, Platteville, CO (US)

(21) Appl. No.: 18/837,098

(22) PCT Filed: Feb. 10, 2022
(86) PCT No.: PCT/US2022/016020

§ 371 Date: Aug. 8, 2024

(51) Int. Cl. A61F 7/08; A61F 7/00; A61F 13/15

(52) U.S. Cl. CPC A61F 7/08; A61F 13/15203; A61F 2007/0054

(57) ABSTRACT

Disclosed herein are systems and methods for providing targeted temperature management (TTM) therapy to a patient. Systems included herein provide an airflow to the patient in additional to a liquid flow that define a thermal energy exchange with the patient. Various systems may provide air at a defined TTM temperature to a thermal contact pad, a mattress or a ventilator that delivers the TTM air to the patient via the ventilation therapy. Also disclosed herein are systems, devices, and methods for preventing, managing, and/or removing perspiration moisture from between the thermal contact pad and the patient. Disclosed herein is a thermal contact pad includes a wicking material to draw moisture away from the patient. Disclosed herein also is a thermal contact pad including airflow that draws moisture away from the patient.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

In some instances of a TTM therapy, the patient may perspire across the contact area of the thermal contact pad causing skin irritation and/or a denaturing of hydrogel. Disclosed herein are systems, devices, and methods for preventing, managing, and/or removing perspiration moisture from the contact area of the thermal contact pad.

In some instances, a patient may benefit by enhancing the thermal energy exchange during a TTM therapy. Typically, the thermal energy exchange is limited by the contact area of the thermal contact pads. Disclosed herein are systems, devises, and methods for providing an airflow to the patient at a defined temperature in accordance with the TTM therapy to enhance the thermal energy exchange with the patient.

### SUMMARY OF THE INVENTION

Briefly summarized, disclosed herein is a medical pad configured to define a thermal energy exchange with a patient, in accordance with some embodiments. The pad includes a fluid containing layer configured for circulation of a targeted temperature management (TTM) fluid therein to define a thermal energy exchange between the TTM fluid and a patient. A plurality of openings extend through the fluid containing layer from a topside to an underside of the fluid containing layer having a wicking material disposed therein, where the wicking material is configured to draw moisture away from a pad contact area of the patient.

In some embodiments, the wicking material extends between the topside and the underside of the fluid containing layer. The openings define a topside opening area and an underside opening area, and in some embodiments, the topside opening area is greater than the underside opening area. In some embodiments, a hydrogel disposed along the underside of the fluid containing layer.

In some embodiments, the pad further includes a cover, where the cover includes a top wall extending over the topside of the fluid containing layer and circumferential wall coupled with the top wall to define a compartment for the fluid containing layer when the pad is applied to the patient. The top wall may include a plurality of vents extending through the top wall and a support structure may be disposed between the top wall and the topside to define a space between the top wall and the top side.

In some embodiments, the pad may include an air pump coupled with the cover, where the air pump is configured to draw air from the compartment to define a vacuum within the compartment. The vacuum may enhance a wicking rate of moisture away from the contact area and the vacuum may further enhance an evaporation rate of moisture away from the wicking material.

Further disclosed herein is a medical system that includes a targeted temperature management (TTM) system. The TTM system includes a TTM module configured to provide a TTM liquid at a defined liquid temperature in accordance with a TTM therapy and a thermal contact pad fluidly coupled with the TTM module, where the pad is configured to receive the TTM liquid from the TTM module and circulate the TTM liquid within a liquid containing layer of the pad to define a thermal energy exchange between the TTM liquid and the patient. The TTM module is further configured to provide a TTM airflow at a defined air temperature to the patient in accordance with the TTM therapy.

The TTM airflow may define a thermal energy exchange between the TTM airflow and the patient and in some embodiments, the temperature of the TTM airflow and the temperature of the TTM liquid are approximately the same.

In some embodiments, the TTM airflow passes through a heat exchanger within the TTM module, where the heat exchanger is configured to transfer heat between the TTM liquid and the TTM airflow and in some embodiments, the TTM module includes an air pump configured to pump the TTM airflow through the heat exchanger.

In some embodiments, the pad is configured to receive the TTM airflow. In some embodiments, the pad includes a plurality of liquid flow channels through which the TTM liquid circulates and a plurality of airflow channels through which the TTM airflow circulates.

In some embodiments, the airflow channels are configured to draw moisture away from a pad contact area of the patient. In some embodiments, the airflow channels include a moisture permeable wall segment extending along an underside of the pad so as to be adjacent the patient's skin, where the moisture permeable wall segment includes one or more of a moisture wicking material or a moisture permeable membrane. In some embodiments, the TTM airflow transfers perspiration moisture away from the patient.

In some embodiments, the medical system further includes a ventilator system fluidly coupled the TTM system, where a ventilation airflow is delivered to the patient in accordance with a ventilation therapy and where the ventilation airflow includes the TTM airflow so that the ventilation airflow defines the thermal energy exchange between the TTM airflow and the patient.

In some embodiments, the medical system further includes a ventilation mattress configured for placement of the patient thereon, where (i) the mattress includes a plurality of mattress airflow channels disposed within the mattress, (ii) the mattress is fluidly coupled with the TTM module so that the TTM airflow passes through the mattress airflow channels, and (iii) the mattress airflow channels are configured within the mattress so that the TTM airflow therethrough defines the thermal energy exchange between the TTM airflow and the patient. In some embodiments, the mattress is configured to conform to a shape of the patient to define a thermal contact between the mattress and the patient. In further embodiments, the mattress includes one or more fastening devices configured to cause portions of the mattress to extend upward adjacent one or more sides of the patient to further define the thermal contact between the mattress and the patient. In some embodiments, the mattress includes a preformed depression configured to receive the patient.

Also disclosed herein is a mattress ventilation system, including a ventilation control module configured to provide a ventilation air at a defined temperature. The ventilation control module includes an air pump configured to cause a flow of the ventilation air, a chiller configured to cool the ventilation air, and a heater to warm the ventilation air.

The system further includes a mattress fluidly coupled with the TTM module, where the mattress is configured to receive the ventilation air from the TTM module. The mattress includes a plurality of airflow channels disposed within the mattress, and the airflow channels are configured within the mattress so that, in use, the flow of ventilation air defines a thermal energy exchange between the ventilation air and a patient placed on the mattress.

In some embodiments, the mattress is configured to conform to a shape of the patient to define a thermal contact between the mattress and the patient. The mattress may also include one or more fastening devices configured to cause portions of the mattress to extend upward adjacent one or more sides of the patient to further define a thermal contact between the mattress and the patient. The mattress may also include a preformed depression configured to receive the patient.

Also disclosed herein is a method of exchanging thermal energy with a patient. The method includes (i) applying a thermal contact pad to the patient; (ii) circulating a liquid from a targeted temperature management (TTM) module within a liquid containing layer of the pad, where the liquid has a defined liquid temperature in accordance with a TTM therapy, and the liquid containing layer defines a topside and an underside; and (iii) providing an airflow to the patient, where the airflow has a defined air temperature in accordance with the targeted temperature management therapy.

In some embodiments of the method, the pad includes a plurality of openings extending through the liquid containing layer from the topside to the underside, where the openings include a wicking material disposed therein, and the method further includes drawing moisture away from the patient through the openings via the wicking material.

In some embodiments of the method, providing the airflow to the patient includes circulating the airflow through air channels of the pad, where the air channels are in fluid communication with a pad contact area of the patient, and the method further includes drawing perspiration moisture away from the patient via the airflow through the air channels.

In some embodiments of the method, providing the airflow to the patient, includes providing ventilation air to the patient via a ventilator, where the ventilation air passes through the TTM module to define the air temperature.

In some embodiments of the method, providing the airflow to the patient includes passing the airflow through a plurality of airflow channels within a mattress for the patient, and in some embodiments, the airflow passes through the TTM module to define the air temperature.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient and a targeted temperature management (TTM) system for cooling or warming the patient, in accordance with some embodiments;

FIG. 2 illustrates a hydraulic schematic of the TTM system of FIG. 1, in accordance with some embodiments;

FIG. 3 illustrates a block diagram depicting various elements of a console of the TTM module of FIG. 1, in accordance with some embodiments;

FIG. 4A is top view of the thermal contact pad of FIG. 1, in accordance with some embodiments;

FIG. 4B is a cross-sectional side view of the thermal contact pad of FIG. 4A cut along sectioning lines 4B-4B, in accordance with some embodiments;

FIG. 5 is a cross-sectional side view of the thermal contact pad of FIG. 4A in combination with a cover extending over the thermal contact pad, in accordance with some embodiments;

FIG. 6A illustrates a patient and a targeted temperature management (TTM) system for cooling or warming the patient where the TTM system further provides a TTM airflow to the thermal contact pad, in accordance with some embodiments;

FIG. 6B illustrates a portion of the hydraulic schematic of FIG. 2 further including an airflow circuit, in accordance with some embodiments;

FIG. 6C is top view of the thermal contact pad of FIG. 6A, in accordance with some embodiments;

FIG. 6D is a cross-sectional side view of the thermal contact pad of FIG. 6C cut along sectioning lines 6D-6D, in accordance with some embodiments;

FIG. 7A illustrates a patient and a medical system that includes the targeted temperature management (TTM) system of FIGS. 1-3 in combination with a ventilator system, in accordance with some embodiments;

FIG. 7B illustrates a portion of the hydraulic schematic of FIG. 2 further including a ventilation airflow circuit, in accordance with some embodiments; and

FIG. 8 illustrates a patient and a medical system that includes the targeted temperature management (TTM) system of FIGS. 1-3 in combination with a mattress ventilation system, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

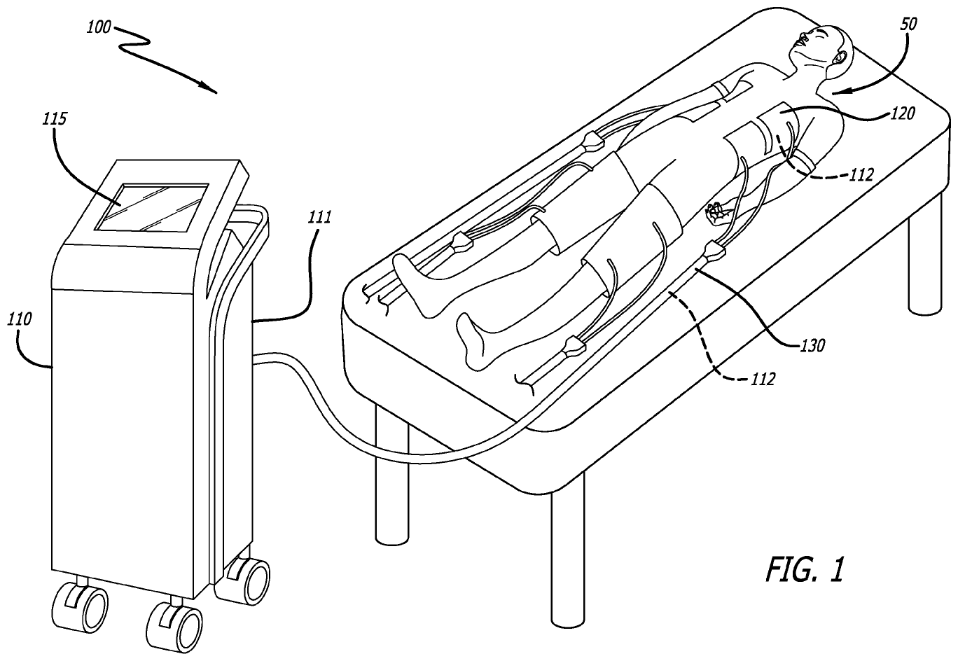

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 includes a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 within the pad 120 to facilitate thermal energy exchange with the patient 50. During the TTM therapy, the TTM module 110 may continually control the temperature of the TTM fluid 112 toward a target TTM temperature. In some instances, the target TTM temperature may change during the TTM therapy.

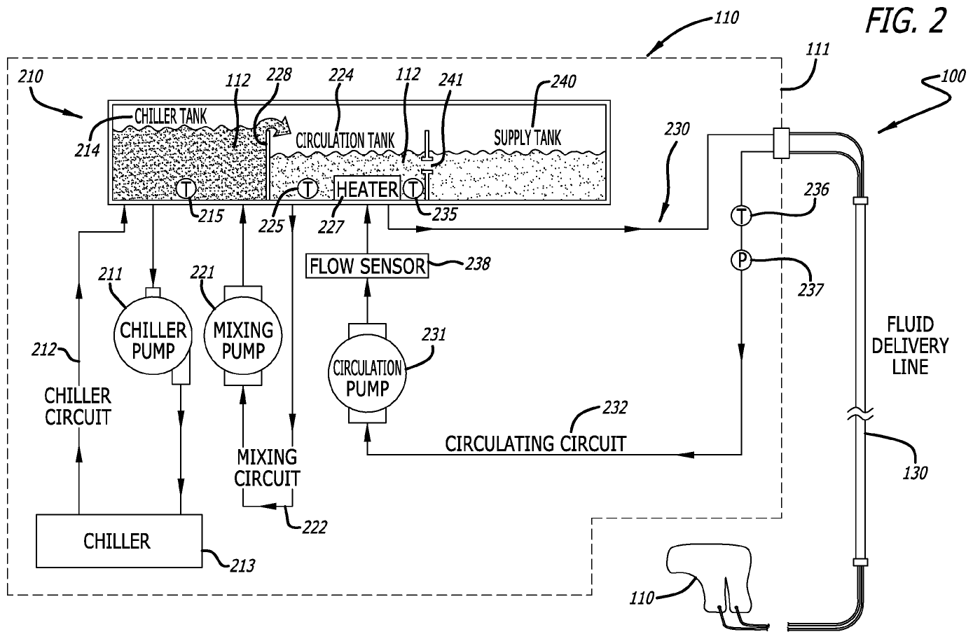

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

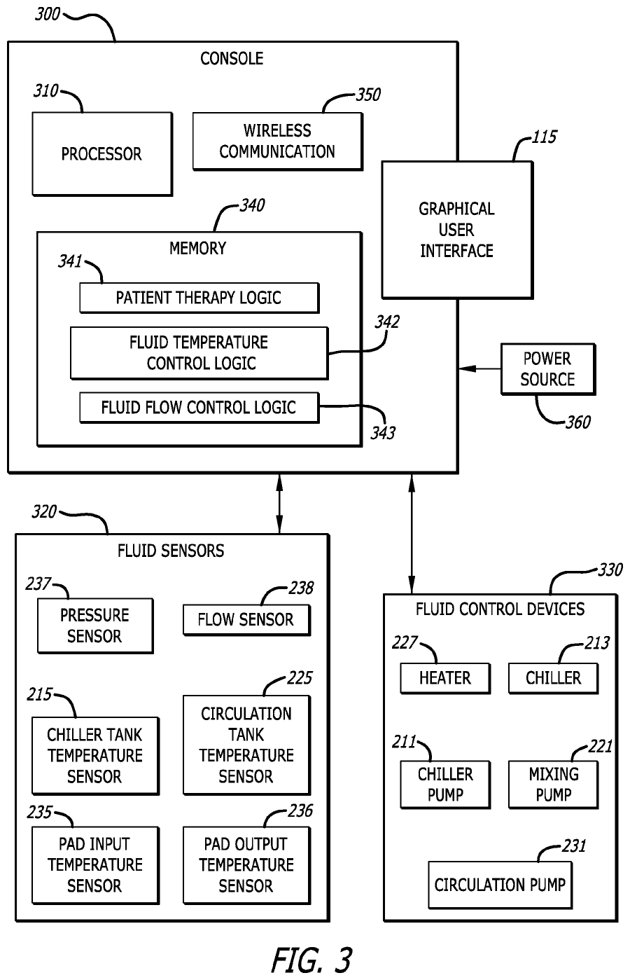

The temperature control subsystem 210 may include a chiller pump 211 to pump (recirculate) TTM fluid 112 through a chiller circuit chiller 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may include may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may receive temperature data from the temperature sensor 215 within the chiller tank and the temperature sensor 225 within the circulation tank 224 and control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 includes a circulation pump 213 to pull TTM fluid 112 from the circulation tank 224 and through a circulating circuit 232 that includes the fluid delivery line 120 and the pad 120 located upstream of the circulation pump 213. The circulating circuit 232 also includes a pressure sensor 237 to represent a pressure of the TTM fluid 112 within the pad 120. The circulating circuit 232 also includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation) the pad 120 so that a pressure within the pad 120 is less than atmospheric pressure (i.e., negative) when fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with a predefined target temperature profile. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may comprise wireless communication capability 350 to facilitate wireless communication with external devices. A power source 360 provides electrical power to the console 300.

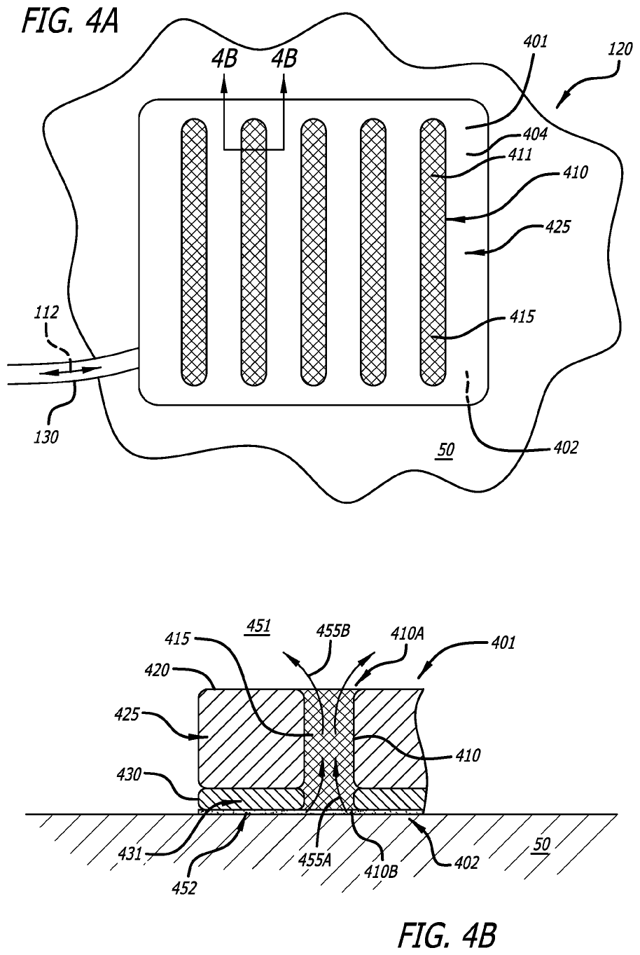

FIG. 4A illustrates a top view of an exemplary pad 120 applied to the patient 50 including the FDL 130 having TTM fluid 112 flowing therethrough to and from the pad 120. The pad 120 defines a topside 401 facing away from the patient 50 and an underside 402 configured for contact, including thermal contact, with the patient 50.

The pad 120 may generally define a rectangular shape. In other embodiments, the pad 120 may define shapes other than rectangular such as circular, oval, or a shape that matches or aligns with the shape of a specific body part. In the illustrated embodiment, the pad 120 generally defines a flat shape in a free state, i.e., absent external force acting thereon. In other embodiments, the pad 120 may define a curved shape in the free state to accommodate a non-flat body part more effectively, such as a leg for, example.

The pad 120 may be configured to accommodate protrusions and/or depressions along a surface of the patient 50. For example, the pad 120 may be structurally flexible in one or more directions to extend over protrusions and/or fill in depressions of the patient surface so that the pad 120 may define a thermal contact with an uneven skin surface the patient. FIG. 4A shows an exemplary arrangement of flow channels 425 extending across the pad 120. The TTM fluid 112 flows along the flow channels 425 to define a heat sink or heat source to the patient 50 in accordance with a temperature of the TTM fluid 112.

The pad 120 includes a plurality of openings 410 extending between the topside 401 and the underside 402 of the pad 120. In some embodiments, the openings 410 may be configured to accommodate a flow of air between the topside 401 and the underside 402 to define a breathability of the pad 120, although breathability is not required. In some embodiments, the openings 410 may also be configured to accommodate a transfer of moisture between the topside 401 and the underside 402 (i.e., wicking of moisture).

The openings 410 may be formed of various shapes as viewed from the topside 401. In some embodiments, the openings 410 may include slots extending between opposite lateral sides of the pad 120. The openings 410 may extend partially across or entirely across the pad 120. In some embodiments, the openings 410 may extend to a perimeter edge of the pad 420. In some embodiments, the openings 410 may include a series of holes (e.g., round holes) arranged randomly or in a defined pattern across the pad 120.

The openings 410 may define an accumulated area 411 comprising a portion of a total area 404 of the pad 120. In some embodiments, the accumulated area 411 may include up to 10 percent, between 10 and 20 percent, between 20 and 30 percent, between 30 and 40 percent, between 40 and 50 percent, or greater than 50 percent of the total area 404.

A material 415 may be disposed within the openings 410. The material 415 may be configured for breathability and/or wicking of moisture. In some instances of a TTM therapy, the patient may perspire across a contact area of a thermal contact pad. The perspiration may cause discomfort to the patient, irritation of the patient's skin, and/or denaturing of a hydrogel disposed across an underside of the pad.

In some embodiments, the material 415 may provide for a breathability of the contact area of the pad 120 to reduce perspiration of the patient 50. In some embodiments, the material 415 may be configured to wick away moisture (e.g., perspiration) from the contact area. In further embodiments, the material 415 may be configured for both breathability and wicking moisture.

A composition of the material 415 may include wool, polyester, polypropylene, nylon, or any other material having wicking characteristics. In some embodiments, the material may be constructed to facilitate moisture wicking due to capillary action of hollows fibers.

FIG. 4B shows a cross-sectional side view of a portion of the pad 120 cut along sectioning lines 4B-4B. The pad 120 may be formed of one or more layers. A fluid containing layer 420 is fluidly coupled to the FDL 130 to facilitate circulation of the TTM fluid 112 within the fluid containing layer 420 along the channels 425. The fluid containing layer 420 having TTM fluid 112 circulating therein defines the heat sink/heat source for the patient 50 in accordance with a temperature of the TTM fluid 112.

The pad 120 may include a thermal conduction layer 430 disposed between the fluid containing layer 420 and the patient 50. The thermal conduction layer 430 is configured to facilitate thermal energy transfer between the fluid containing layer 420 and the patient 50 via heat conduction. The thermal conduction layer 430 may be attached to the fluid containing layer 420 along an underside of the fluid containing layer 420. The thermal conduction layer 430 may be conformable to provide for intimate contact with the patient 50. In other words, thermal conduction layer 430 may conform to a contour of the patient 50 to inhibit space or air pockets between the thermal conduction layer 430 and the patient 50. In some embodiments, the thermal conduction layer 430 may include a hydrogel 431.

As illustrated in FIG. 4B, the opening 410 extends between a topside opening 410A and an underside opening 410B. In some embodiments, the topside opening 410A and the underside opening 410B may be similar in shape and size (e.g., area). In other embodiments, the topside opening 410A and the underside opening 410B may be different in shape and size to optimize, breathability, moisture wickability, and/or thermal energy exchange. For example, the underside opening 410B may be sized to enable ingress 455A of moisture 452 (perspiration from the patient 50) into the material 415 while minimizing any loss of thermal energy exchange via the thermal conduction layer 430. Similarly, the topside opening 410A may be maximized in size to facilitate evaporation 455B of the moisture 452 from the material 415 to the environment 451. As such, the topside opening 410A may be greater in size/area than the underside opening 410B. In some embodiments, natural convection currents of the air adjacent the topside 401 of the pad 120 due to a temperature difference between the fluid containing layer 420 and the environment 451 may be utilized to enhance the evaporation 455B of the moisture 452 from the material 415 to the environment 451.

In some embodiments, the pad 120 may be compressible such that a volume of the openings 410 changes in response to movement of the patient 50. The change in volume may define a pumping action to enhance the ingress 455A of moisture 452 into the material 415 from the patient 50 and egress of moisture 452 from the wicking material 415 to the environment 451 via evaporation 455B.

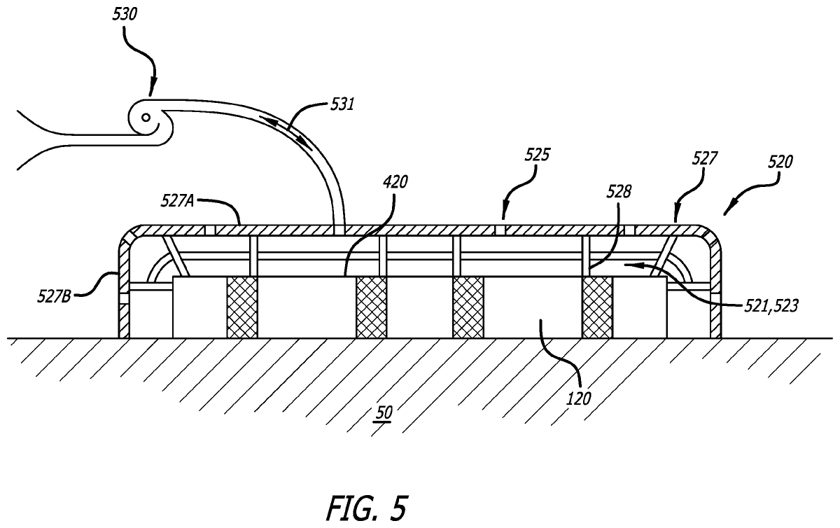

FIG. 5 illustrates a cover 520 that may be placed over the pad 120 during use of the pad 120. In some embodiments, the pad 120 may include the cover 520, i.e., the cover 520 may be attached to the pad 120. The cover 520 may enhance moisture evaporation from the pad 120. The cover 520 may define a space 521 between the topside 401 of the pad 120 and the cover 520. The cover 520 may be formed of a sheet material such as flexible fabric material, for example.

In some embodiments, the cover 520 may include a support structure 528 to prevent collapse of the cover 520, i.e., maintain the space 521. The space 521 in combination with a plurality of vents 525 extending through a cover wall 527, may facilitate airflow along the topside 401 including airflow defined by natural convection currents. The cover wall 527 may include a top wall 527A and a circumferential side wall 527B.

In some embodiments, the cover 520 may extend downward adjacent a circumference of the pad 120 to the skin surface of the patient 50 to define a compartment 523 enclosing the pad 120. In further embodiments, the cover 520 may include an air pump 530, such as a fan, for example. The air pump 530 may facilitate a forced airflow into or out of the compartment 523 to define a forced air current adjacent the topside 401 of the pad 120 to enhance an evaporation rate of the moisture 452 (see FIG. 4B). In some embodiments, the air pump 530 may define a negative pressure within the compartment 523 and the negative pressure may further enhance the wicking rate of moisture from the patient and/or the evaporation rate of the moisture 452.

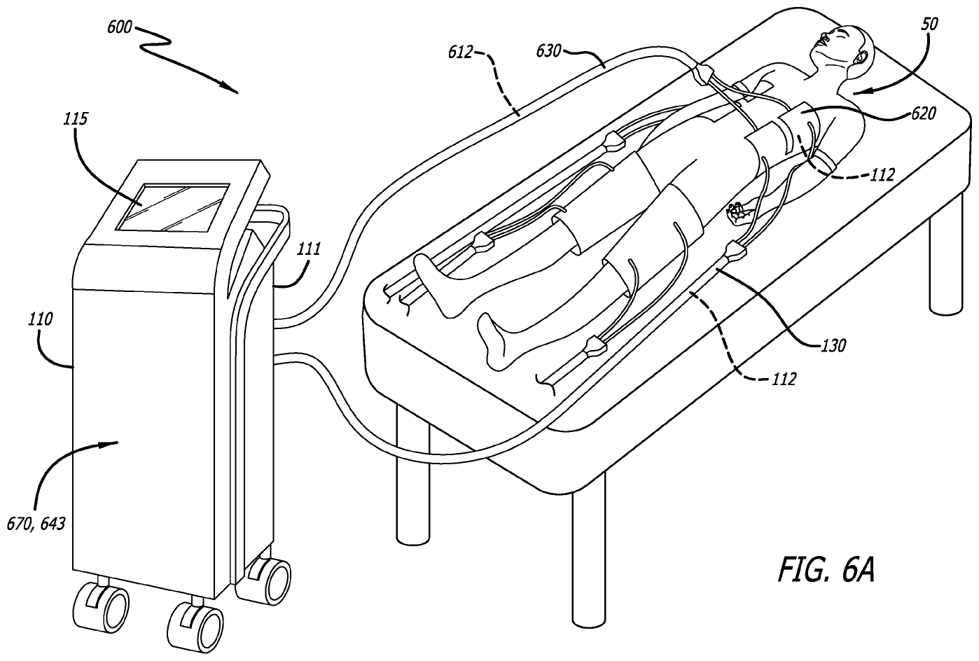

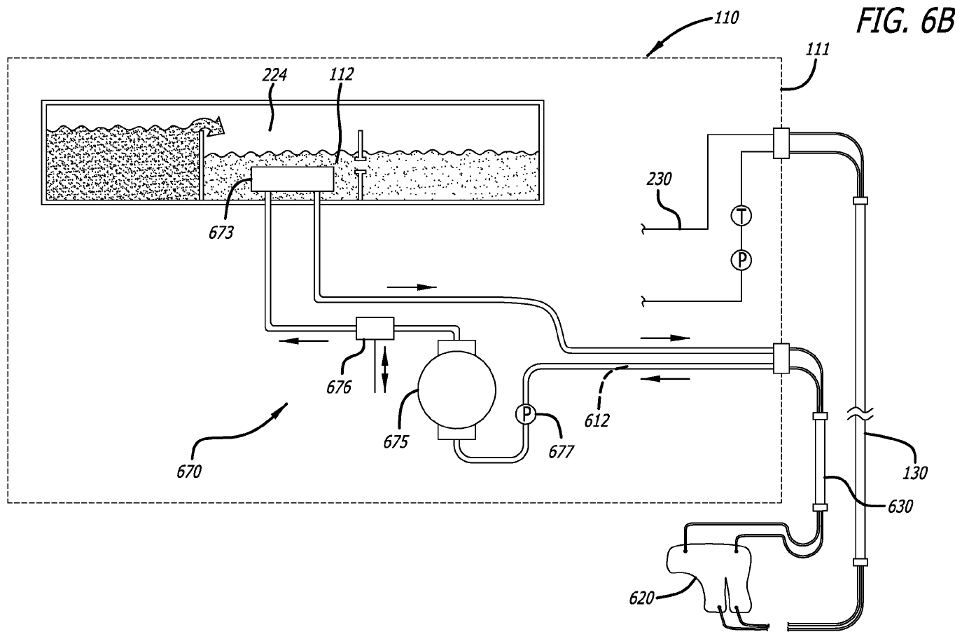

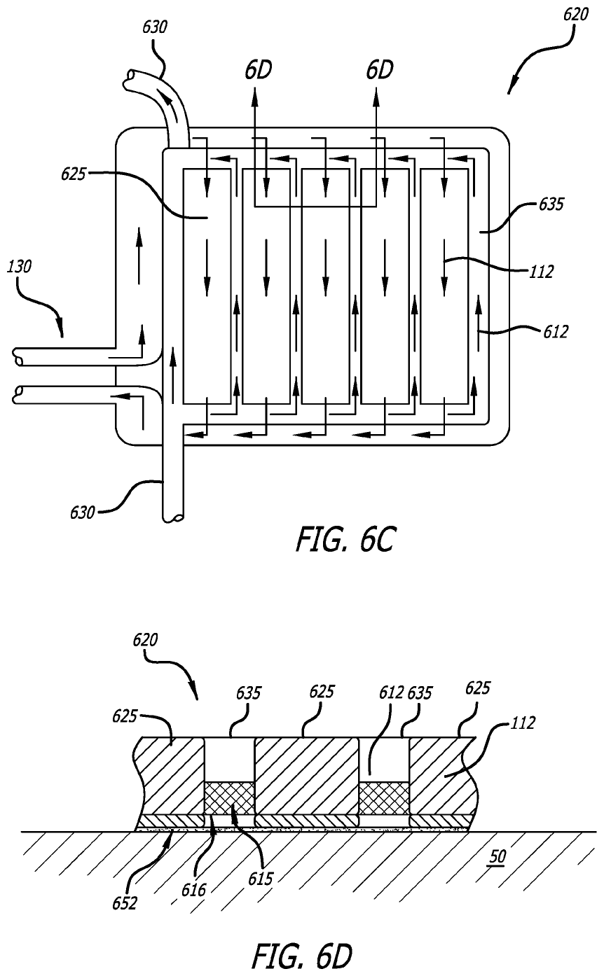

FIG. 6A illustrates second embodiment of a TTM system 600. The system 600 may resemble the TTM system 100 of FIGS. 1-3 in some respects including functionality, fluid components, and console components. The system 600 includes the system module 110 that further includes an airflow circuit 670 and air control logic 643. The system 600 also includes a thermal contact pad 620. In some embodiments, the system 600 may include multiple pads 620. The system 600 includes the fluid delivery line 130 coupled with the system module 110 having TTM fluid 112 flowing therethrough. The system 600 further includes an air delivery line 630 having air 612 flowing therethrough between the airflow circuit 670 housed within the system module 110 and the pad 620. The airflow control logic 643 is included in the system module 110 as a component of the console 300 (FIG. 3).

FIG. 6B illustrates a portion of the hydraulic schematic of FIG. 2 further including the airflow circuit 670 incorporated into the system module 110 and housed within the module housing 111. The airflow circuit 670 generally includes an air pump 675, a heat exchanger 673, and a pressure regulator 676. The air pump 675 is configured to pump the air 612 through the airflow circuit 670 including to and from the pad 620 via the air delivery line 630. The pressure regulator 676 may be located at any location along the airflow circuit 670 and in some embodiments, the pressure regulator 676 is configured to cause the air pressure to be negative (i.e., a vacuum) within the pad 620. In some embodiments, the pressure regulator 676 may be provide for air to flow into and/or out off the airflow circuit 670. More specifically, the pressure regulator 676 may include an air pump to transfer air 612 into or out of the airflow circuit 670. In some embodiments, the airflow circuit 670 may include a pressure sensor 677. In some embodiments, one or more of the air pump 675, the pressure regulator 676, or the pressure sensor 677 may be communicatively coupled with the console 300 so that the air control logic 643 may define active control of the air flow rate and/or the air pressure.

The heat exchanger 673 is coupled with the circulating circuit 232 (see FIG. 2) of the TTM module 110. In some embodiments, the heat exchanger 673 may be a liquid-to-air heat exchanger coupled between the TTM fluid 112 and the air 612 so that the temperature of the air 612 leaving the heat exchanger 673 is the same or similar to the temperature of the TTM fluid 112. Although not required, the heat exchanger 673 may be located within the circulation tank 224 of the TTM module 110.

FIG. 6C is a top view of the pad 620 illustrating an exemplary flow schematic of the TTM fluid 112 and the air 612. FIG. 6D illustrates a cross-sectional view of a portion of the pad 620 as cut along section lines 6D-6D. The pad 620 exchanges TTM fluid 112 with the TTM module 110 via the FDL 130 and the pad 620 exchanges air 612 with the TTM module 110 via the air delivery line 630. The TTM fluid 112 circulates within the pad 620 via a series of flow channels 625. Similarly, the air 612 flow through the pad 620 via a series of flow channels 635. The flow channels 625 and the flow channels 635 may define any suitable arrangement extending across the pad 620. The structure of the pad 620, or more specifically the structure of the channels 625, 635, may be configured to accommodate a negative pressure of the TTM fluid 112 and the air 612 without collapsing. As the air 612 and the TTM fluid have a similar temperature, the thermal exchange with the patient 50 may be defined by both the temperature of the air 612 and the temperature of the TTM fluid 112.

FIG. 6D illustrates a cross-sectional view of a portion of the pad 620 as cut along section lines 6D-6D where the pad 620 is applied to a patient 50. As discussed above, in some instances of a TTM therapy, the patient may perspire across a contact area of a thermal contact pad. The perspiration may cause discomfort to the patient, irritation of the patient's skin, and/or denaturing of a hydrogel disposed across an underside of the pad. The flow of air 612 through the channels 635 facilitate a reduction in perspiration of the patient 50. The flow of air 612 through the channels 635 facilitate a reduction in accumulated perspiration moisture 652 between the pad 620 and the patient's skin.

In some embodiments, a material 615 may be disposed within the channels 635. The material 615 may be configured for breathability and/or wicking moisture 652. As shown, in some embodiments, the material 615 may extend across only a portion of a cross-sectional area of the channel 635 so that the air 612 may flow freely through the portion of the cross-sectional area of the channel 635 unoccupied by the material 615. In other embodiments, the material 615 may extend across an entire cross-section of the channel 635 and the air 612 may flow through the material 615. In some embodiments, a material 615 may provide for a breathability of the contact area of the pad 120 to reduce perspiration of the patient 50. In some embodiments, the material 615 may be configured to wick away moisture 652 (e.g., perspiration) from contact area. In further embodiments, the material 615 may be configured for both breathability and wicking moisture.

A composition of the material 615 may include wool, polyester, polypropylene, nylon, or any other material having wicking characteristics. In some embodiments, the material may be constructed to facilitate moisture wicking due to capillary action of hollows fibers.

In some embodiments, the pad may include a semipermeable membrane 616 in addition to or in lieu of the material 615. The membrane 616 may be disposed along an underside of the channel 635 so as to be disposed between the air 615 and patient's skin. The membrane 616 may allow for the transfer of moisture 652 across the membrane 616. In some embodiments, the membrane 616 may be omitted so that the material 615 may contact the skin directly.

In use, the flow of air 612 may draw moisture 652 away from the patient's skin. More specifically, perspiration of the patient 50 may pass through the membrane 616 and the material 615 to the air 612. The air 612 may cause an enhanced evaporation rate of the moisture 652 from the membrane 616 and/or material 615 to the air 612. In some embodiments, the material 615 and membrane 616 may both be omitted so that the air 616 flows in direct contact with the patient's skin.

In some embodiments, the pad 620 may be compressible such that the flow area of the channels 635 decrease and increase in response to movement of the patient 50. The decrease and increase of the flow area of the channels 635 may define a pumping action to enhance the ingress of moisture 652 into the wicking material 615 through the membrane 616 and egress of moisture 652 from the wicking material 615 via the air 612.

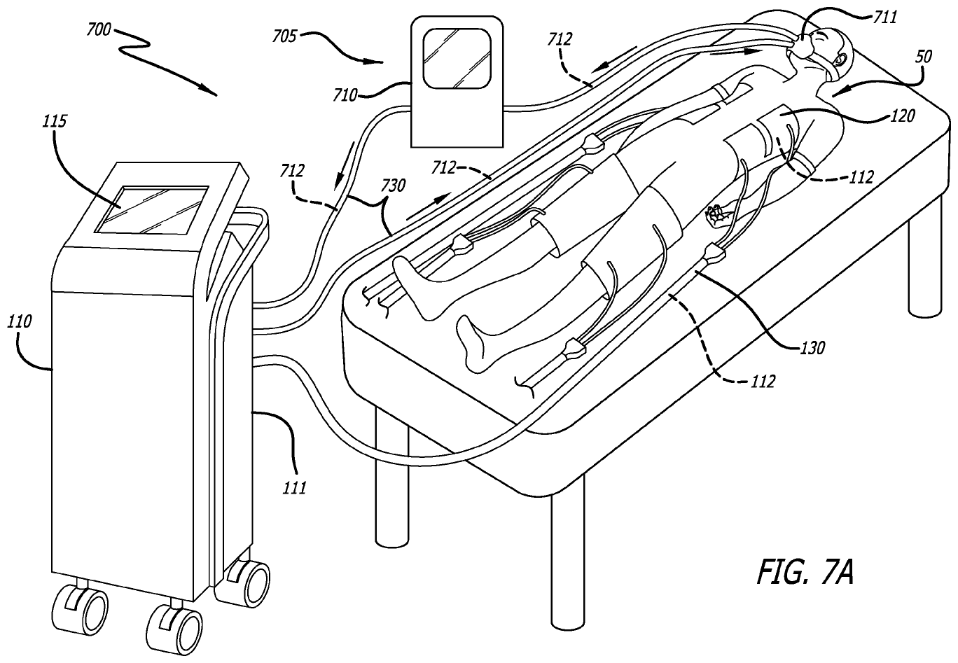

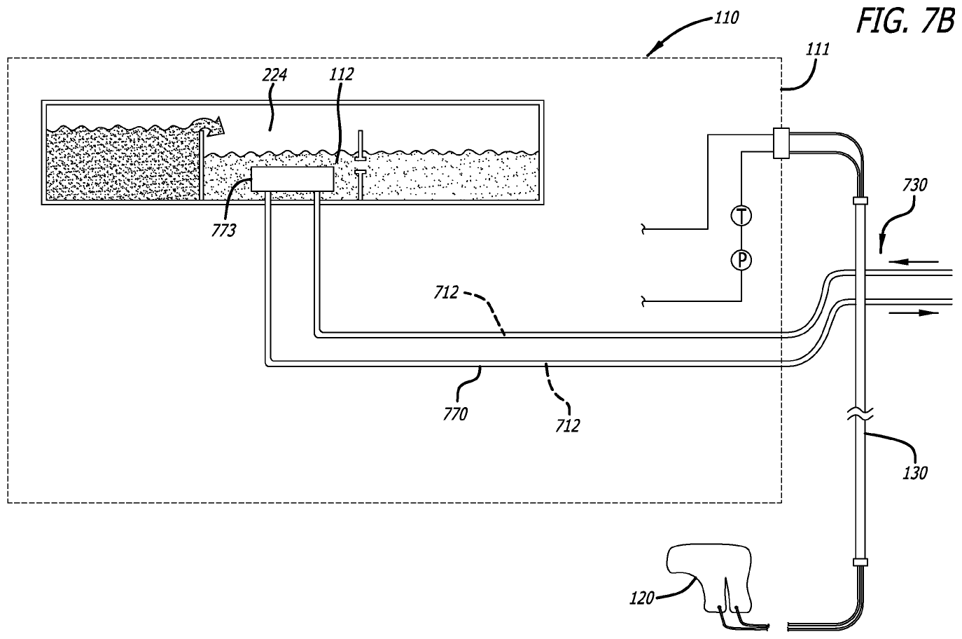

FIG. 7A illustrates a medical system 700. The system 700 generally includes the TTM system 100 of FIGS. 1-3 coupled with a ventilator system 705 that includes a ventilator module 710, ventilator tubing 730, and a ventilator mask 711. The system 700 is configured to enhance the TTM therapy by defining the temperature of the ventilation air 712 in coordination with the temperature of the TTM fluid 112. An airflow circuit 770 is incorporated into the TTM module 110 and is connected to the ventilator tubing 730 so that air 712 from the ventilator module 710 flows through the airflow circuit 770 prior to flowing to the patient 50.

FIG. 7B illustrates a portion of the hydraulic schematic of FIG. 2 further including the airflow circuit 770 incorporated into the system module 110 and housed within the module housing 111. The airflow circuit 770 generally includes a heat exchanger 773 coupled with the circulating circuit 232 (see FIG. 2) of the TTM module 110. In some embodiments, the heat exchanger 773 may be a liquid-to-air heat exchanger coupled between the TTM fluid 112 and the air 712 so that the temperature of the air 712 leaving the heat exchanger 773 is the same or similar to the temperature of the TTM fluid 112. Although not required, the heat exchanger 773 may be located within the circulation tank 224 of the TTM module 110, as shown.

In use, the ventilator module 110 prepares the air 712 in accordance with a ventilation therapy for the patient 50. The TTM module 110 further prepares the air 712 (i.e., defines the temperature of the air 712) to assist the TTM therapy, i.e., enhance the thermal energy exchange. More specifically, the air 712 from the ventilator module 710 flows through the TTM module 110 (i.e., the heat exchanger 773) and then flows to the patient 50. The air 712 further flows from the patient 50 back to ventilator module 710 to complete the ventilation circuit.

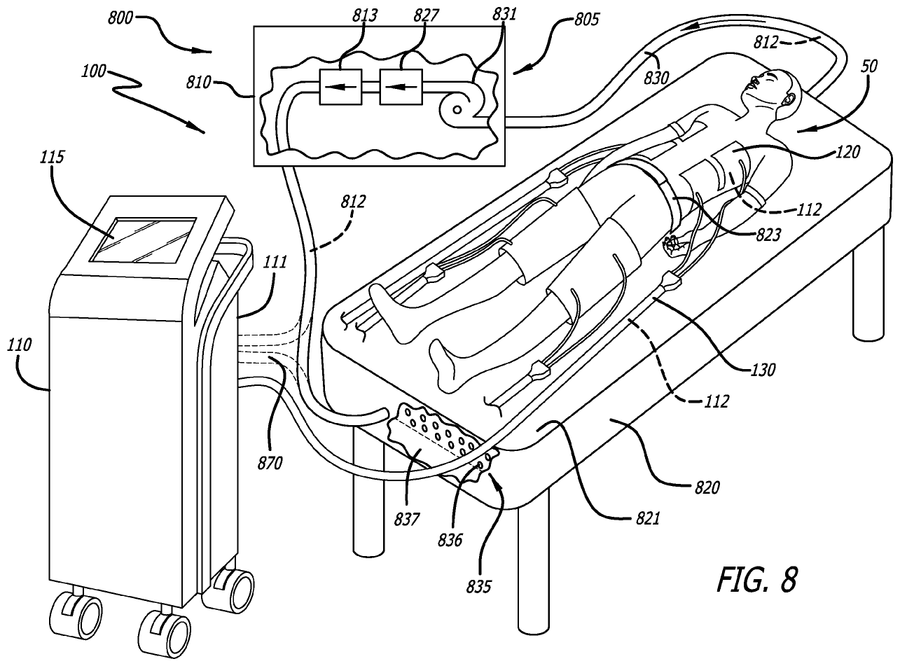

FIG. 8 illustrates a TTM system 800 that includes the TTM system 100 and a mattress ventilation system 805. In some embodiments, the mattress ventilation system 805 may be a stand-alone system separate from the TTM system 100. The mattress ventilation system 805 is generally configured to facilitate thermal energy exchange between a mattress 820 and the patient 50 in accordance a temperature of air flowing through the mattress 820. The system 805 generally includes a ventilation module 810 coupled with the mattress 820 via an air delivery line 830. The ventilation module 810 generally includes an air pump 831, a heater 827, and a chiller 813 so that the ventilation module 810 may provide warm or cool air to the mattress 820. The air pump 831 causes air 812 to flow through the heater 827 and/or the chiller 813 to increase or decrease the temperature of the air 812, respectively. The air pump 831 further causes air 812 to flow to and from the mattress 820 via the air delivery line 830. The ventilation module 810 may further include components (not shown) for controlling the temperature of the air 812, such as temperature sensors, microprocessors, logic, and power converters, for example.

In some embodiments, the mattress ventilation system 805 may be coupled with the TTM system 100. More specifically, the air delivery line 830 may be connected with an airflow circuit 870 incorporated into the TTM module 110. The airflow circuit 870 may in some respects resemble the airflow circuit 770 of FIG. 7B. As such, the airflow circuit 870 may entirely or partially define the temperature of the air 812. In such embodiments, the heater 827 and/or the chiller 813 may be omitted from the ventilation module 810, i.e., the mattress ventilation system 805 may rely solely on the TTM module 110 to define the temperature of the air 812.

In further embodiments, the airflow circuit 770 may resemble the airflow circuit 670 of FIG. 6B. In such embodiments, ventilation module 810 module may be omitted completely. More specifically the TTM module 110 may define the flow of the air 812 to and from the mattress 820 in combination with defining the temperature of the air 812.

FIG. 8 shows a cut away portion of the mattress 820 illustrating plurality of channels 835 disposed within the mattress 820 through which the air 812 may flow. The channels 835 may define any suitable arrangement across the mattress 820 to establish a temperature of the mattress 820 along a top surface 821 thereof so that mattress 820 may facilitate thermal exchange between the patient 50 and the air 812.

In some embodiments, mattress 820 may include an air circulation layer 836 containing the channels 835 and a cushion layer 837 disposed along an underside of the circulation layer 836. The circulation layer 837 may be constructed to prevent collapsing of the channels 835 when the patient 50 is placed on the mattress 820. The circulation layer 837 may be sufficiently flexible to conform to the shape of the patient 50 such as partially wrapping around the legs or torso of the patient 50. In some embodiments, a pressure of the air 812 within the channels 835 may contribute to a conformability of the mattress 820, similar to an air mattress, for example.

The cushion layer 837 may be configured to define support for the circulation layer 836. In some embodiments, the cushion layer 837 may be constructed of a compressible foam material so that a weight of the patient 50 may define a depression in the cushion layer 837 to enhance the conformability of the mattress 820 to the patient 50. In some embodiments, the mattress 820 may define a flat top surface 821 in a free state, i.e., absent the patient 50. In other embodiments, the mattress 820 may define a preformed shape (e.g., a depression) to enhance the conformability of the mattress 820 to the patient 50.

In some embodiments, the mattress 820 may include one or more fastening devices 823 (e.g., straps) to secure the mattress 820 or portions thereof to the patient 50. The fastening devices 823 may be configured to alter the shape of the top surface 821 to cause portions of the mattress to partially wrap around patient 50 (e.g., extend upward along a side of the patient 50). For example, a strap may cause portions of the mattress to partially wrap around a leg or the torso of the patient to enhance a contact area of the mattress 820 with the patient 50 thereby enhancing thermal exchange.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A medical pad, comprising:
a fluid containing layer configured for circulation of a targeted temperature management (TTM) fluid therein to define a thermal energy exchange between the TTM fluid and a patient;
a plurality of openings extending through the fluid containing layer from a topside to an underside of the fluid containing layer; and
a wicking material disposed within the openings, the wicking material configured to draw moisture away from a pad contact area of the patient.

2. The pad of claim 1, wherein the wicking material extends between the topside and the underside.

3. The pad of claim 1, wherein:
the openings define a topside opening area and an underside opening area, and
the topside opening area is greater than the underside opening area.

4. The pad of claim 1, further comprising a hydrogel disposed along the underside of the fluid containing layer.

5. The pad of claim 1, further comprising a cover comprising:
a top wall extending over the topside of the fluid containing layer, and
circumferential wall coupled with the top wall to define a compartment for the fluid containing layer when the pad is applied to the patient.

6. The pad of claim 5, wherein the top wall comprises a plurality of vents extending through the top wall.

7. The pad of claim 5, wherein the cover further comprises a support structure disposed between the top wall and the topside to define a space between the top wall and the top side.

8. The pad of claim 5, further comprising an air pump coupled with the cover, wherein the air pump is configured to draw air from the compartment to define a vacuum within the compartment.

9. The pad of claim 8, wherein the vacuum enhances a wicking rate of moisture away from the contact area.

10. The pad of claim 8, wherein the vacuum enhances an evaporation rate of moisture away from the wicking material.

11. The pad of claim 1, wherein:
the pad is compressible such that a volume of the openings changes in response to movement of the patient, and
the volumetric change enhances the wicking rate of moisture from the contact area and the evaporation rate of moisture away from the wicking material.

12. A medical system, comprising:
a targeted temperature management (TTM) system comprising:
a TTM module configured to provide a TTM liquid at a defined liquid temperature in accordance with a TTM therapy;
a thermal contact pad fluidly coupled with the TTM module, the pad configured to:
receive the TTM liquid from the TTM module, and
circulate the TTM liquid within a liquid containing layer of the pad to define a thermal energy exchange between the TTM liquid and the patient,
wherein the TTM module is further configured to provide a TTM airflow at a defined air temperature to the patient in accordance with the TTM therapy.

13. The system of claim 12, wherein the temperature of the TTM airflow and the temperature of the TTM liquid are approximately the same.

14. The system of claim 12, wherein in use, the TTM airflow defines a thermal energy exchange between the TTM airflow and the patient.

15. The system of claim 12, wherein the TTM airflow passes through a heat exchanger within the TTM module, the heat exchanger configured to transfer heat between the TTM liquid and the TTM airflow.

16. The system of claim 15, wherein the TTM module comprises:
an air pump configured to pump the TTM airflow through the heat exchanger.

17. The system of claim 12, wherein the pad is configured to receive the TTM airflow.

18. The system of claim 12, wherein the pad comprises:
a plurality of liquid flow channels through which the TTM liquid circulates, and
a plurality of airflow channels through which the TTM airflow circulates.

19. The system of claim 18, wherein the airflow channels are configured to draw moisture away from a pad contact area of the patient.

20. The system of claim 18, wherein the airflow channels include a moisture permeable wall segment extending along an underside of the pad so as to be adjacent the patient's skin.

21. The system of claim 20, wherein the permeable wall segment includes one or more of a moisture wicking material or a moisture permeable membrane.

22. The system of claim 19, wherein in use, the TTM airflow transfers perspiration moisture away from the patient.

23. The system of claim 12, further comprising:
a ventilator system fluidly coupled the TTM system,
wherein a ventilation airflow delivered to the patient in accordance with a ventilation therapy includes the TTM airflow so that the ventilation airflow defines the thermal energy exchange between the TTM airflow and the patient.

24. The system of claim 12, further comprising:
a ventilation mattress configured for placement of the patient thereon, wherein:
the mattress comprises a plurality of mattress airflow channels disposed within the mattress,
the mattress is fluidly coupled with the TTM module so that the TTM airflow passes through the mattress airflow channels, and
the mattress airflow channels are configured within the mattress so that the TTM airflow therethrough defines the thermal energy exchange between the TTM airflow and the patient.

25. The system of claim 24, wherein the mattress is configured to conform to a shape of the patient to define a thermal contact between the mattress and the patient.

26. The system of claim 24, wherein the mattress includes one or more fastening devices configured to cause portions of the mattress to extend upward adjacent one or more sides of the patient to define the thermal contact between the mattress and the patient.

27. The system of claim 24, wherein the mattress includes a preformed depression configured to receive the patient.

28-37. (canceled)
